(19) United States
(12) Patent Application Publication
(10) Pub. No.: US 2024/0138740 A1
(43) Pub. Date: May 2, 2024

(54) METHOD AND DEVICE FOR DETECTING A REPRESENTATIVE CARDIAC ELECTRICAL ACTIVITY

(71) Applicants: UNIVERSITE DE BORDEAUX, BORDEAUX (FR); CENTRE HOSPITALIER UNIVERSITAIRE DE BORDEAUX, TALENCE (FR); FONDATION BORDEAUX UNIVERSITE, BORDEAUX (FR)

(72) Inventors: Laura BEAR, TALENCE (FR); Olivier BERNUS, LACANAU DE MOIS (FR); Rémi DUBOIS, MERIGNAC (FR); Michel HAISSAGUERRE, TALENCE (FR); Nolwenn TAN, LIBOURNE (FR)

(21) Appl. No.: 18/550,616

(22) PCT Filed: Mar. 25, 2022
(86) PCT No.: PCT/EP2022/058005

§ 371 Date: Sep. 14, 2023

(30) Foreign Application Priority Data
Mar. 25, 2021 (FR) FR2103044

(51) Int. Cl. A61B 5/318; A61B 5/00; A61B 5/08; G16H 40/63

(52) U.S. Cl. CPC A61B 5/318; A61B 5/0806; A61B 5/6831; G16H 40/63

(57) ABSTRACT

A method for detecting a representative cardiac activity includes the arrangement of a plurality of electrodes, the measurement of the value of electrophysiological descriptors, and the calculation (CALC) of a score.

### FIELD OF THE INVENTION

The field of the invention relates to methods and devices for generating an electrophysiological parameter related to an individual's cardiac activity. More particularly, the field of the invention relates to methods implemented by means of surface electrodes recording signals used to detect a representative cardiac electrical activity.

### PRIOR ART

Currently, there are means for analyzing signals coming from surface electrodes in order to identify an electrical activity of the myocardium. This analysis makes it possible to prevent certain cardiac pathologies by identifying singular electrical activities within the myocardium.

These means, even though very reliable, only focus on a limited number of cardiac activity indicators derived from the analysis of cardiac electrical signals. For example, the duration of the QRS (duration of ventricular depolarization) is frequently used as an indicator of cardiac pathologies. This measurement is a very good indicator for detecting certain singular activities of the heart, but this measurement alone is only an outline of certain cardiac characteristics. For example, it is possible for a patient with a given pathology that the measurement of QRS duration may be representative of an electrophysiological singularity. However, this measurement may prove to be insufficient to characterize certain electrophysiological activities of an individual, notably an electrophysiological activity that can be used or corroborated with other variables to anticipate a cardiac risk.

Thus, by focusing on few parameters, it is common not to detect characteristic electrophysiological activities.

Currently known methods have the drawback, by focusing on a very limited number of indicators, of offering only a very limited view of a patient's cardiac activity.

To date, existing solutions aim to confirm or detect the presence of a singular electrical activity. However, this activity usually remains at a low level or drowned in noise or even masked by a larger electrical pattern such as a QRS complex. No solution makes it possible, from a plurality of surface electrodes, to determine the characteristics of a source of a singular electrical activity or even to know a wide range of electrophysiological variables of a patient, which are capable of forming a corpus of singularities that can be used for medical purposes or to prevent a cardiac risk.

The invention therefore aims to propose a method for measuring the cardiac activity of a patient that addresses the aforementioned drawbacks.

### SUMMARY OF THE INVENTION

According to one aspect, the invention relates to a method for generating an electrophysiological parameter which comprises:
selection of a subset of electrophysiological descriptors from a set of predefined electrophysiological descriptors according to an input parameter defining a measurement context, each electrophysiological descriptor of the subset being associated with at least one channel, a signal type, a signal marker and a statistical modality for calculation;
arrangement of a plurality of surface electrodes on a patient's body;
recording of a plurality of cardiac electrical activities defining said channels, each channel being obtained by the recordings of at least two electrodes;
estimation of the set of electrophysiological descriptors of the subset, each electrophysiological descriptor being calculated from the statistical modality that is applied to the signal marker of the acquired signal according to the signal type on a selected channel associated with said electrophysiological descriptor;
comparison of the value of the set of electrophysiological descriptors with at least one threshold value specific to the set of electrophysiological descriptors, said at least one threshold value being defined by a statistical distribution of said descriptors of a set of patients of a reference group;
calculation of a score defining an electrophysiological parameter as a function of the exceeding of at least one threshold value defined by the statistical distribution.

One advantage of the invention is to propose a method taking into account a set of different electrophysiological descriptors to characterize a cardiac activity. The possibility of selecting the measurement context by taking descriptors that concern areas on the patient's body, types of signals analyzed, signal markers and different statistical modalities allows a composite measurement of the patient's cardiac activity. The fact of composing the score after exceeding a threshold for a plurality of descriptors with different modalities makes it possible to effectively detect cardiac activities that may be characteristic. Finally, this method makes it possible to form a corpus of electrophysiological variables making it possible to characterize a singular activity of an individual's cardiac activity.

“Patients of a reference group” is taken to mean a group of individuals selected from a class of individuals. The class of individuals is selected from one or more criteria. The criteria are selected from:
a type of person considered;
the geographical area of residence or origin of the persons considered;
the age of the persons considered;
the presence of a pathology in the persons considered; and/or
the morphology of the persons considered.

For example, for a patient with a cardiac pathology, it is possible that the measured indicator (e.g. the QRS duration) is normal, whereas it is considered a characteristic measurement of this pathology. In the case of the invention, since a plurality of descriptors is used, even if one of them gives a value that is within the norm, the other descriptors used can provide an alert to the presence of a characteristic cardiac activity. Thus, there is much greater effectiveness in the detection of said characteristic activity, compared to known methods.

According to one embodiment, at least one descriptor of the subset being associated with a statistical modality different to that of a second descriptor of the subset and a signal marker different to that of the second descriptor. This provision makes it possible to obtain a subset which comprises several descriptors bearing different physiological information from each other.

The different steps of the method according to the invention may be implemented by calculation means such as calculators. The latter may be those of an electronic board of dedicated equipment or those of a remote computer or data server.

According to one embodiment, the comparison step is performed by comparing the value of the set of descriptors with the threshold value defined by a statistical distribution of said descriptors of the set of healthy patients of dimension equal to the number of descriptors of the subset. This provision makes it possible to define a relevant threshold value as a function of a set of healthy patients.

According to one embodiment, the comparison step is performed by comparing the value of each electrophysiological descriptor with at least one threshold value specific to said electrophysiological descriptor, said at least one threshold value being defined by a statistical distribution of said descriptor of a set of healthy patients; and the step of calculating the score defining the electrophysiological parameter is performed by incrementing said score each time an electrophysiological descriptor exceeds at least one threshold value specific to it. This characteristic makes it possible to define the calculation of the score by comparing the values with a control population. The calculated score is therefore representative of the patient's cardiac activity.

According to one embodiment, for at least one electrophysiological descriptor, the at least one channel is derived from a predefined area of the patient's body from a set of predefined areas. This provision makes it possible to take into account the location of measurements on the patient's body, the areas being predefined.

According to one embodiment, the subset comprises at least one second geographical descriptor which is associated with several channels and several geographical groups, each geographical group being formed by a central channel and the at least four channels adjacent to the central channel, the value of the electrophysiological descriptor being determined:
by comparing the value, for each geographical group, of the measurement of each channel according to the signal type and the signal marker selected with at least one geographical threshold value specific to said electrophysiological descriptor and said channel; and
by counting the number of geographical groups for which the value of at least three channels exceeds the geographical threshold value that is specific to it.

This provision makes it possible to have a second type of descriptors which take into account geographical groups. The addition of this other type of descriptors makes it possible to take into account the concentration of singular measured values and enriches the subset of selected descriptors.

According to one embodiment, the input parameter defining the measurement context defines a subset of characteristic descriptors of a given cardiac pathology. This characteristic makes it possible to adapt the subset of descriptors to a cardiac pathology that it is wished to analyze.

According to one embodiment, the input parameter defining the measurement context defines a subset of descriptors characteristic of a patient profile comprising at least one age and/or gender/sex. Taking these characteristics into account makes it possible to increase the accuracy of the electrophysiological parameter generated.

According to one embodiment, each first electrophysiological descriptor of the subset is associated with at least one channel of a predefined area of the patient's body from a set of predefined areas and in that for each electrophysiological descriptor, the predefined area on the patient's body is chosen from:
An upper right area of the torso;
An upper left area of the torso;
A lower right area of the torso;
A lower left area of the torso; and
The entire torso of the patient.

This provision makes it possible to have descriptors that take into account the geographical location on the patient's body of the measurements taken. Thus, there is a variety of descriptors that take into account several types of information.

According to one embodiment, the plurality of electrodes comprises at least fifteen electrodes, preferably twenty-five. This provision makes it possible to obtain a mesh of measurement electrodes sufficiently fine to obtain the electrophysiological parameter.

According to one embodiment, for each electrophysiological descriptor, the signal type analyzed is chosen from:
A unipolar signal taken between an electrode of the chosen body area and a reference electrode;
A vertical bipolar signal taken between two electrodes of the given area, one of the two electrodes being offset along a vertical line with respect to the other electrode;
A horizontal bipolar signal taken between two electrodes of the given area, one of the two electrodes being offset along a horizontal line with respect to the other electrode; and
A Laplacian signal estimated by subtracting from the potential of a central electrode the average tension of the eight electrodes directly adjacent to said central electrode.

This provision makes it possible to take into account several types of signals and therefore to have descriptors that take into account several types of signal measurement. Thus, the descriptors take into account several types of information and report more faithfully on the condition of said patient.

According to one embodiment, at least one reference electrode arranged on a lower limb or an upper limb of the patient. This provision makes it possible to measure a reference potential.

According to one embodiment, a plurality of reference electrodes is arranged on the surface of the patient's lower or upper limb(s). This provision makes it possible to obtain a reference potential with high precision.

According to one embodiment, for at least one descriptor, the signal marker is the voltage measurement of an averaged signal. The averaging of the signal makes it possible to obtain a stable measurement of said voltage.

According to one embodiment, for at least one descriptor, the signal marker is the measurement, on the averaged and filtered signal between 40 and 250 Hertz, of the duration of depolarization of the ventricles or fragmentation of the signal during depolarization of the ventricles. The duration of ventricular depolarization is a very representative measurement of cardiac activity.

According to one embodiment, for at least one descriptor, the signal marker is the measurement on the discrete wavelet decomposition of the signal:
Of the energy of the sum of the wavelets;
Of the Kurtosis;
Of the Fisher asymmetry coefficient; or
Of the number of local minima.

This provision enables different types of signals all bearing different information enriching the selected subset to be taken into account.

According to one embodiment, for at least one descriptor, the signal marker is the measurement on the continuous wavelet decomposition of the signal of the number of local maxima chains. This measurement is a measurement that makes it possible to report a singular cardiac activity.

According to one embodiment, for at least one descriptor, the signal marker is the measurement on the wavelet taken between 256 and 512 Hertz of the signal:
Of the Kurtosis; or
Of the number of areas with reduced amplitudes.

These measurements make it possible to report measurements on the curve such as the flattening thereof.

According to one embodiment, for at least one descriptor, the signal marker is the measurement on the wavelet taken between 128 and 256 Hertz of the signal:
Of the Kurtosis;
Of the number of areas of reduced amplitude; or
Of the RMS (Root Mean Square).

These measurements make it possible to report measurements on the curve such as the flattening thereof.

According to one embodiment, for at least one descriptor, the signal marker is the measurement on the wavelet taken between 64 and 128 Hertz of the RMS (Root Mean Square). This measurement is used to report the patient's cardiac activity.

According to one embodiment, for each first descriptor, the statistical modality is chosen from:
The fifth percentile minimum of the measured values of the signal on each electrode in the predefined area;
The ninety fifth percentile maximum of the measured values of the signal on each electrode of the predefined area;
The average of the measured values of the signal on each electrode of the predefined area;
The standard deviation of the measured values of the signal on each electrode of the predefined area;
The median of the measured values of the signal on each electrode of the predefined area; and
The interquartile of the measured values of the signal on each electrode of the predefined area.

This provision makes it possible to select a statistical modality that makes it possible to process the multiple measurements made while choosing the statistical modality that is relevant, according to whether it is wished to analyze maxima or minima for example. These modalities also make it possible to avoid extreme aberrant measurements.

According to one embodiment, the estimation of electrophysiological descriptors is performed during a low respiration phase of the patient. This provision makes it possible to perform the measurement during a respiration phase that does not disturb the measurement.

According to one embodiment, a plethysmography belt is arranged on the patient to estimate the respiration phases thereof.

According to one embodiment, the method of the invention may comprise a prior step aimed at selecting a subset of electrophysiological descriptors characteristic of a cardiac electrical activity.

For example, this prior step may be carried out by a method for selecting a subset of first electrophysiological descriptors characteristic of a characteristic cardiac electrical activity from a set of first predefined descriptors. Each first electrophysiological descriptor is associated with at least one channel, one signal type, one signal marker and one statistical modality for calculation. The method comprises a recording of a plurality of electrical activities defining said channels and comprises:
Estimation, for a first set of patients not having a predefined condition, of values of each descriptor of the set of predefined descriptors;
Estimation, for a second set of patients having the predefined condition, of values of each descriptor of the set of predefined descriptors;
Generation of a first vector characteristic of the condition of each patient of the first and second sets of patients in which each component corresponds to a condition relative to the predefined condition;
Generation of a descriptor vector for each descriptor in a metric space in which each component corresponds to the value of the descriptor for each patient;
First quantification for each descriptor of a first proximity factor between the values of the components of the first characteristic vector and the values of the components of the descriptor vector;
First selection and inclusion in the subset of at least one descriptor having optimal quantified proximity factor values;
Second quantification:
   of a second proximity factor between the values of the components of each descriptor not selected during the first selection step and the values of the components of the descriptor vector(s) selected during the first selection step; and
   of a third proximity factor between the values of the components of each descriptor not selected during the first selection step and the characteristic vector component values;
Second selection and inclusion in the subset of at least one new descriptor as a function of the value of the second proximity factor and the value of the third proximity factor quantified during the step of second quantification.

According to one aspect, the invention relates to a device or a system comprising means for implementing the method of the invention. The means may comprise calculators, memories, electronic boards, electrodes and electrode holders. The device or the system of the invention may comprise computers or servers when calculation resources are required. The invention is hereafter described such that the described characteristics may relate to the method of the invention or to the device or the system of the invention.

The invention also relates to a device for generating an electrophysiological parameter which comprises:
a plurality of surface electrodes configured to be placed on a patient's body and to measure an electrical potential of the surface of the patient's body, each surface electrode defining a channel;
a means of measuring the signal of each channel;
a calculation means configured to:
   select a subset of descriptors from a set of predefined descriptors according to an input parameter defining a measurement context, each electrophysiological descriptor of the subset being associated with at least one channel, a signal type, a signal marker and a statistical modality for calculation; at least one descriptor of the subset being associated with a statistical modality different to that of a second descriptor of the subset and a signal marker different to that of the second descriptor;
   record a plurality of cardiac electrical activities defining said channels, each channel being obtained by the recordings of at least two electrodes;
   estimate the set of electrophysiological descriptors of the subset, each electrophysiological descriptor being calculated from the statistical modality that is applied to the signal marker of the acquired signal according to the signal type on a selected channel associated with said electrophysiological descriptor;
   compare the value of the set of electrophysiological descriptors with at least one threshold value specific to the set of electrophysiological descriptors, said at least one threshold value being defined by a statistical distribution of said descriptors of a set of patients;
   calculate a score defining an electrophysiological parameter as a function of the exceeding of the at least one threshold value defined by the statistical distribution.

According to one embodiment, the electrodes are arranged on the surface of the patient using adhesive strips. This provision allows a practical and quick layout of the adhesive strips on the patient.

According to one embodiment, the device comprises a device for detecting a patient's respiration phases, preferably a plethysmography belt. This provision makes it possible to perform the measurement during a respiration phase that does not disturb the measurement.

According to one embodiment, the device is capable of implementing the method according to the invention.

### BRIEF DESCRIPTION OF THE FIGURES

Other characteristics and advantages of the invention will become clearer upon reading the following detailed description, in reference to the appended figures, that illustrate:

FIG. 1: a schematic flowchart of the method according to the invention;

FIG. 2: a flowchart presenting the selection of a descriptor;

FIG. 3: a front view of the torso of a patient on which is arranged a plurality of measuring electrodes to implement the method according to the invention;

FIG. 4: a view of a plurality of measuring electrodes in a predefined area of the patient's body;

FIG. 5: two curves illustrating the method for calculating an asymmetry index;

FIG. 6: a graph illustrating the method for calculating a Kurtosis index;

FIG. 7: a view of a curve illustrating the method for calculating the number of areas of reduced amplitude.

### DESCRIPTION OF THE INVENTION

According to a first aspect, the invention relates to a method for generating an electrophysiological parameter. The term “electrophysiological parameter” is taken to mean a datum derived from an electrical measurement which is characteristic of a patient's physiological activity.

The invention also relates to a device for generating an electrophysiological parameter. The device according to the invention is capable of implementing the method mentioned previously. Subsequently, the elements described in this description will be applicable both to the method according to the invention and to the device according to the invention.

The method according to the invention will be described in support of FIG. 1, which is a schematic flowchart of the method according to the invention.

According to a first aspect, the invention relates to a method for generating an electrophysiological parameter. A first step in this method is a step of selecting a subset of first descriptors {Dₖ} from a set of predefined descriptors {D_{N}}. “Electrophysiological descriptor Dᵢ” is taken to mean a context of measurement of an electrical datum associated with an activity detected on the surface of a patient. Each first electrophysiological descriptor Dᵢ is associated with the recording of at least one channel Vᵢ. The recording of a channel Vᵢ corresponds to the recording of the electrical activity captured by at least one electrode EL arranged on the surface of a patient's body. Each first descriptor Dᵢ is associated with at least one channel Vᵢ on a predefined area Zᵢ on the patient's body. “Predefined area Zᵢ” is taken to mean the area of the patient's body on which are deposited the measuring electrode(s) EL, the electrical activities of which are recorded to obtain the channel(s) Vᵢ. Thus, the surface of a patient's body may be segmented into different functional and/or geometrical and/or physiological areas. These areas may therefore correspond to geographical areas on the patient's body, to functional areas in relation to the patient's cardiac activity, or to areas corresponding to the patient's physiology and/or physiology.

Each first descriptor Dᵢ is associated with a signal type Tᵢ measured on the selected channels Vᵢ. For example, signal type Tᵢ is taken to mean the measurement of a tension between two electrodes. The different types of signals Tᵢ that can be selected will be described later.

Each first descriptor Dᵢ is associated with a signal marker Mᵢ. “Signal marker Mᵢ” is taken to mean the characteristic of the signal type Tᵢ that will be measured. A set of signal markers Mᵢ used in the context of the invention comprises the measurement of frequency and energy characteristics of the signal. The different signal markers Mᵢ that may be selected are described below.

Each first descriptor Dᵢ is associated with a statistical modality MSᵢ. “Statistical modality MSᵢ” is taken to mean a statistical measurement modality applied to the measurements made on the signals. For example, a statistical modality MSᵢ may be the calculation of the average of the signal marker Mᵢ measured on several electrodes. The different statistical modalities MSᵢ that may be selected will be described later.

Each first electrophysiological descriptor Dᵢ is therefore defined by both a selection of the predefined area Zᵢ of the patient's body, the signal type Tᵢ, the signal marker Mᵢ and the statistical modality MSᵢ.

In the method according to the invention, an input parameter Inp is acquired. The input parameter Inp defines a measurement context. “Measurement context” is taken to mean the number of descriptors Dᵢ that will be selected as well as, for each descriptor Dᵢ, the combination of the predefined area Zᵢ, the signal type Tᵢ, the signal marker Mᵢ and the statistical modality MSᵢ that are selected.

According to a first example, the input parameter is generated from a patient profile, for example defined by his or her age, gender, medical history, and possibly other parameters.

According to a second example, the input parameter is generated from a selection of a predefined pathology. The pathology may be associated in a database or memory with a set of descriptors. This case may be interesting to associate the measured electrical activity with said selected pathology.

According to a third example, the input parameter is defined by selecting a set of descriptors. This selection may be made by an operator from a user interface.

According to a fourth example, the input parameter is defined by selecting a set of electrodes. This selection may be made by an operator from a user interface.

According to a fifth example, the input parameter is defined by a set point emitted by a computer-implemented device or method. The set point may correspond to a set of descriptors selected according to an algorithm configured according to different parameters. Such an algorithm is for example described in the application FR2103047 filed on 25 Mar. 2021, the title of which is “METHOD FOR SELECTING ELECTROPHYSIOLOGICAL DESCRIPTORS”.

According to an example, the input parameter is defined by a combination of these different alternatives.

According to one embodiment, at least two first descriptors Dᵢ, Dⱼ of the subset {Dₖ} are associated with a different statistical modality MSᵢ. In other words, two of the first descriptors Dᵢ, Dⱼ are not associated with the same statistical modality MSᵢ.

At least two first descriptors Dᵢ, Dⱼ of the subset {Dₖ} are associated with a different signal marker Mᵢ. In other words, two of the first descriptors Dᵢ, Dⱼ are not associated with the same signal marker Mᵢ.

A plurality of surface electrodes EL is arranged on the patient's body. Thus, at least two electrodes are placed on the patient's skin. These electrodes EL are configured to collect an electrical potential on the surface of the patient's skin.

A recording ENR of a plurality of cardiac electrical activities defining said channels Vᵢ is performed. During this step, it is preferable to record all the available channels. In other words, the electrical activity of each electrode of the plurality of electrodes arranged on the patient's body is recorded. For the purposes of the recordings, the invention may be implemented notably by means of a memory making it possible to record the data acquired and/or the data processed by one of the steps of the method of the invention.

An estimation EST of the set of electrophysiological descriptors Dᵢ of the subset of descriptors {Dₖ} is made. In this step of the method, each value of each descriptor Dᵢ of the subset {Dₖ} is calculated. The calculation is performed for each descriptor Dᵢ in the predefined area Zᵢ, with the signal type Tᵢ, on the signal marker Mᵢ, and according to the statistical modality MSᵢ that are associated with it.

The calculated values of each descriptor Dᵢ are then compared to a threshold value V_{threshold}. Each threshold value V_{threshold} is characteristic of the electrophysiological descriptor Dᵢ in question. Thus, there is a threshold value V_{threshold} specific to each descriptor Dᵢ.

A score is then calculated from the number of descriptors Dᵢ of which the value has exceeded the threshold value V_{threshold} specific to it. Exceeding the threshold value V_{threshold} is taken to mean exceeding by a higher value or by a lower value. The direction in which the threshold is exceeded is specific to each descriptor Dᵢ. Each time the threshold value V_{threshold} is exceeded by a descriptor Dᵢ, the score is incremented.

Finally, a score is obtained that defines an electrophysiological parameter.

The method according to the invention thus makes it possible to obtain an electrophysiological parameter that takes into account the calculation of the value of several descriptors Dᵢ which have measurement and calculation modalities that are very different. Thus, the electrophysiological parameter does not take into account only one parameter to calculate the score defining the electrophysiological parameter. Since each descriptor is a bearer of information on the cardiac activity of the patient that is specific thereto, the method according to the invention makes it possible to generate the electrophysiological parameter the most representative of the patient's cardiac activity.

Alternatively, the comparison step may be performed taking into account the statistical distribution of the set of descriptors in a space having a dimension equal to the number of descriptors Dᵢ of the subset {Dₖ}. According to this alternative, the set of descriptors is compared to a threshold value derived from the multi-dimensional distribution.

Electrophysiological Descriptors

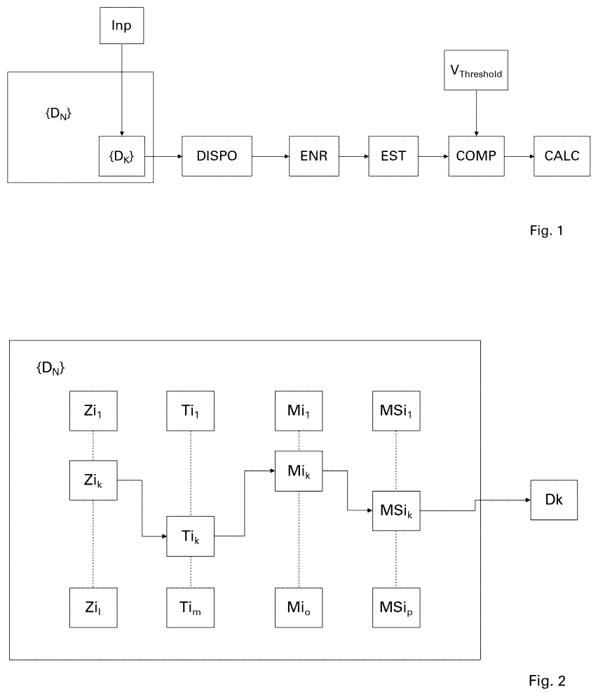

As described previously, each electrophysiological descriptor Dᵢ is associated with at least one channel Vᵢ of a predefined area Zᵢ of the patient's body, a signal type Tᵢ, a signal marker Mᵢ, and a statistical modality for calculation MSᵢ. These four elements will be described in detail below. The selection of a descriptor Dᵢ from the set of descriptors {D_{N}} is illustrated in FIG. 2, which is a flowchart showing the method for selecting a descriptor Dᵢ. To select a descriptor Dᵢ, a selection is made of a predefined area Zᵢ from a set of predefined areas Zᵢ. A selection is made of a signal type Tᵢ from a set of signal types Tᵢ. A selection is made of a signal marker Mᵢ from a set of signal markers Mᵢ. A selection is made of a statistical modality MSᵢ from a set of statistical modalities MSᵢ.

Patient's Body Area

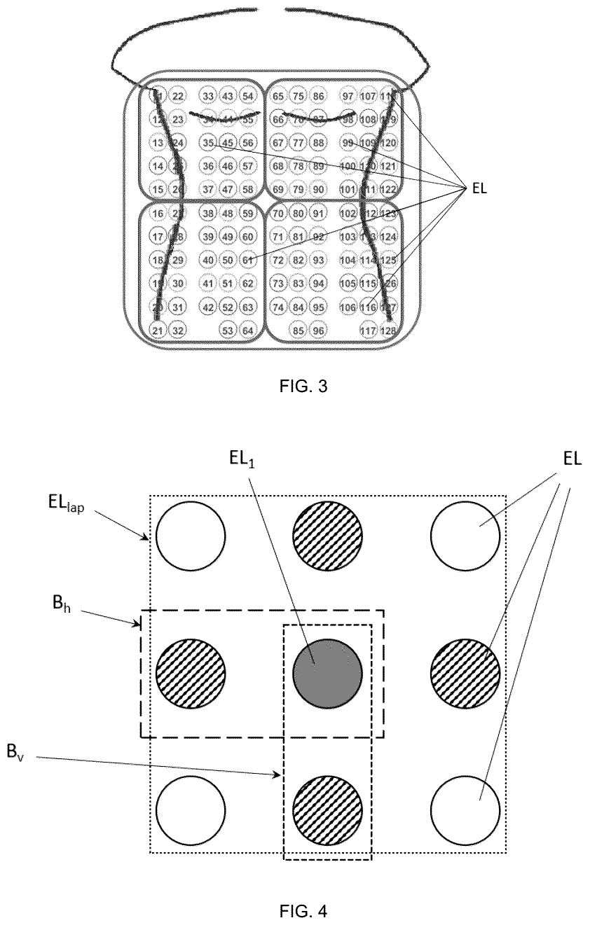

As shown in FIG. 3, a plurality of electrodes EL are placed on the surface of the patient's torso. The number of electrodes in the example shown may vary, for example the number of electrodes may be much lower than that shown, or much higher. The plurality of electrodes EL covers a large part of the patient's torso. As can be seen, this plurality of electrodes is separated into four distinct areas on it. A first part of the electrodes EL is located on an upper right area Zᵢ of the patient's torso. A second part of the electrodes EL is located on an upper left area Z₂ of the patient's torso. A third part of the electrodes EL is located on a lower right area Z₃ of the patient's torso. A fourth part of the electrodes EL is located on a lower left area Z₄ of the patient's torso. Finally, the plurality of electrodes EL is comprised in an area comprising the totality Z₅ of the patient's torso.

The demarcation between the areas situated to the left of the torso and those situated to the right of the torso is a vertical line passing through the center of the torso, or substantially through the center of the torso.

The demarcation between the areas situated on the lower torso and those situated on the upper torso is a horizontal line through the center of the torso.

Each area comprises a predefined number of electrodes EL. The number of electrodes arranged per area may be of the order of thirty or so. For example, there may be thirty electrodes EL per area.

Advantageously, each area comprises the same number of electrodes EL. Obviously, the area comprising the entire torso Z₅ comprises a different number of electrodes than the others, because this area Z₅ comprises the reunion of the electrodes of all the other areas Z₁, Z₂, Z₃ and Z₄.

Hereafter, when mention is made of the selection of electrodes in an area, the selection of one or more electrodes EL in said area will be meant.

Alternatively, a lower number of electrodes EL may be provided, for example nine electrodes EL per area Z₁, Z₂, Z₃, and Z₄. Thus, in this case, the area Z₅ comprising the entire torso of the patient comprises thirty six electrodes EL.

Each channel Vᵢ is obtained by recording the electrical activity of at least two electrodes EL. These at least two electrodes may be two electrodes from one or more areas of the patient's torso. These at least two electrodes may also be an electrode from an area of the patient's torso and a reference electrode.

Geographical Groups

According to one embodiment, the subset {D_{N}} comprises at least one second geographical electrophysiological descriptor Dᵢ. The at least one geographical descriptor Dᵢ is associated with at least one channel Vᵢ and with several geographical groups. A geographical group is formed by an electrode EL and the four electrodes EL that are situated directly next to it. A geographical group is shown in FIG. 3. This geographical group comprises a central electrode EL₁ and the electrode situated directly above it. It also comprises the electrode situated directly below the central electrode EL₁, the electrode situated directly to the left of the central electrode EL₁, and the electrode situated directly to the right of the central electrode EL₁. These four electrodes are shown hatched in FIG. 3. For the second geographical descriptors, the measurement of the value according to the signal type and the signal marker is performed on all the available geographical groups of the plurality of electrodes arranged on the patient's body. Other provisions of electrodes may be envisaged. It is notably possible to select the central electrode and the four electrodes situated at the top left, top right, bottom left and bottom right of the central electrode EL₁. These are the electrodes that appear without a pattern in FIG. 3. More electrodes may also be provided, for example nine electrodes in the geographical group. These are the nine electrodes EL in FIG. 3 for example.

For each geographical group, the value obtained for each electrode EL of said group is compared with at least one geographical threshold value. The at least one geographical threshold value is obtained from a statistical distribution of the value of the considered electrode EL of a set of healthy patients. When at least three electrode values of a geographical group exceed their geographical threshold value, then the geographical group is considered as significant. Finally, the value of the descriptor is the number of significant geographical groups counted. Alternatively, a geographical group may be considered as signifying from two electrodes exceeding their geographical threshold value, or instead with four electrodes.

It may be noted that in the case of a second geographical descriptor, the statistical modality is not taken into account, the value of the descriptor being the number of geographical groups detected.

Advantageously, the subset {Dₖ} of descriptors comprises at least one first descriptor Dᵢ and at least one second geographical descriptor. Additionally, the subset {Dₖ} comprises several first descriptors Dᵢ. According to this alternative, the subset {Dₖ} comprises a second geographical descriptor per signal marker used in the first descriptors Dᵢ of the subset {Dₖ}.

Signal Type

Each electrophysiological descriptor Dᵢ is associated with a signal type Tᵢ. In this section, reference will be made to FIG. 4, which is a schematic representation of nine contiguous electrodes EL on the patient's body. In this figure, each circle represents an electrode EL. The dotted areas represent the different electrodes EL selected in the different signal types.

The signal type Tᵢ may preferably be chosen between four different signal types.

A first signal type Tᵢ is a unipolar signal. A unipolar signal is a signal taken between an electrode EL of the predefined area Zᵢ and a reference electrode. In other words, the unipolar signal type is the tension measured between the electrode of the predefined area and the reference electrode. “Reference electrode” is taken to mean an electrode that is not situated in one of the areas of the patient's torso defined previously. For example, a reference electrode may be an electrode placed on a lower limb or an upper limb of a patient.

A second signal type Tᵢ is a vertical bipolar signal. A vertical bipolar signal is a signal taken between an electrode in the predefined area and the electrode situated directly below it on the patient's torso. In other words, the signal type acquired is the tension between the two electrodes EL. A vertical bipolar signal is taken between two electrodes of the predefined area Zᵢ. These two electrodes form a vertical bipole Bᵥ.

A third signal type Tᵢ is a horizontal bipolar signal. A horizontal bipolar signal is a signal taken between an electrode in the predefined area and an electrode situated directly next to it along a horizontal line on the patient's torso. In other words, the signal type acquired is the tension between the two electrodes EL. The horizontal bipolar signal is taken between two electrodes of the predefined area Zᵢ. These two electrodes form a horizontal bipole Bₕ.

“A vertical axis” is taken to mean an axis defined by a line parallel to the axis of the body taken in its largest dimension. “A horizontal axis” is taken to mean an axis defined in a plane perpendicular to the vertical axis and tangential to the surface of the human body.

According to one embodiment, a frame of reference linked to the human body may be defined in such a way as to define the vertical and horizontal axes notably with respect to the morphology of the human body or other reference axes.

According to one embodiment, the invention may be defined with respect to other reference axes than the vertical axis and the horizontal axis insofar as the positions of the electrodes may be defined in the position indicator related to the human body.

A fourth signal type Tᵢ is a Laplacian signal. A Laplacian signal is estimated by subtracting from the potential of a central electrode EL₁ the average of the potentials of the eight electrodes that are directly adjacent to said central electrode. In other words, the Laplacian signal is a tension composed between the central electrode EL₁ and a set of electrodes EL peripheral to the central electrode EL₁. These nine electrodes form a Laplacian electrode ELₗₐₚ.

Signal markers Each electrophysiological descriptor Dᵢ is associated with a signal marker Mᵢ. A signal marker Mᵢ is a modality of measuring a physical quantity associated with the types of signals measured by the electrodes EL.

The signal marker Mᵢ associated with a descriptor Dᵢ is preferably chosen from fourteen signal markers Mᵢ. These signal markers Mᵢ are described below.

A first signal marker Mᵢ corresponds to the measurement of an averaged electrical signal. Averaged electrical signal is taken to mean the calculation, made on the measured tension, of the average between the maximum peak and the minimum peak of the QRS. This measurement of QRS duration is generally quite representative of a cardiac activity.

Two signal markers which are measured on the filtered signal between 40 and 150 Hertz will now be described. The signal is filtered using a bandpass filter. Advantageously, the bandpass filter is a bidirectional Butterworth filter. A bidirectional Butterworth filter has the advantage of limiting oscillations due to filtering, which makes the calculation of values for certain signal markers Mᵢ more accurate.

A signal marker Mᵢ on the filtered signal is the QRS duration on the filtered signal. To measure this value, a position indicator is placed at the start of the QRS and a second mark is placed at the end of the QRS. The time between the two marks is measured. This operation may be performed automatically thanks to a QRS start and end detection algorithm. Alternatively, this duration may be measured manually by an operator on an interface. An automatic measurement of the QRS duration and a manual check of said measurement by the operator on the interface may also be provided.

According to one embodiment, the QRS duration is detected by moving a sliding window measuring the energy of the filtered signal. When an energy threshold is exceeded, a position indicator is placed that marks the start of the window. The QRS end position indicator is placed in the same way.

Another signal marker Mᵢ is the measurement of the fragmentation of the filtered averaged signal between 40 Hertz and 250 Hertz. According to this marker Mᵢ, the number of QRS peaks on the filtered signal is measured. Peak is taken to mean a local maximum of the filtered signal curve. The number of peaks is measured on the section of the curve corresponding to the QRS. The start and end marks of the QRS are set in the same way as for the previous marker Mᵢ, which as a reminder is the QRS duration marker on the filtered signal.

The following markers Mᵢ are calculated on the decomposition into wavelets of the signal. For these markers Mᵢ, either continuous wavelet decomposition or discrete wavelet decomposition may be used.

The four markers Mᵢ shown below are calculated on the discrete wavelet decomposition.

A first marker Mᵢ is the calculation of the energy on the discrete wavelet decomposition of the signal. Specifically, the energy is calculated on the sum of the coefficients over several levels. Typically, the sum of the coefficients is made between 64 Hertz and 1024 Hertz, i.e. on the four levels of this frequency band. According to one embodiment, the measured energy is normalized with respect to the QRS duration. Alternatively or additionally, the energy is normalized with respect to the maximum signal amplitude.

A second marker Mᵢ calculated on the discrete wavelet transform is the measurement of the so-called Kurtosis index Sₖᵤ. “Kurtosis” is taken to mean an index making it possible to estimate the spread of a given curve. FIG. 6 illustrates several measurements of curve spread on three exemplary curves. For a flat curve P, the Kurtosis index is negative. For a slender curve E, the index is positive. Thus, the more the curve is spread, the more negative the Kurtosis. When the curve is narrow, the Kurtosis is positive. The Kurtosis of a curve representing a normal distribution N is equal to zero. Specifically, the Kurtosis Sₖᵤ is calculated on the sum of the coefficients over several levels of the discrete wavelet decomposition. Typically, the sum of the coefficients is made between 64 Hertz and 1024 Hertz, i.e. on the four levels of this frequency band.

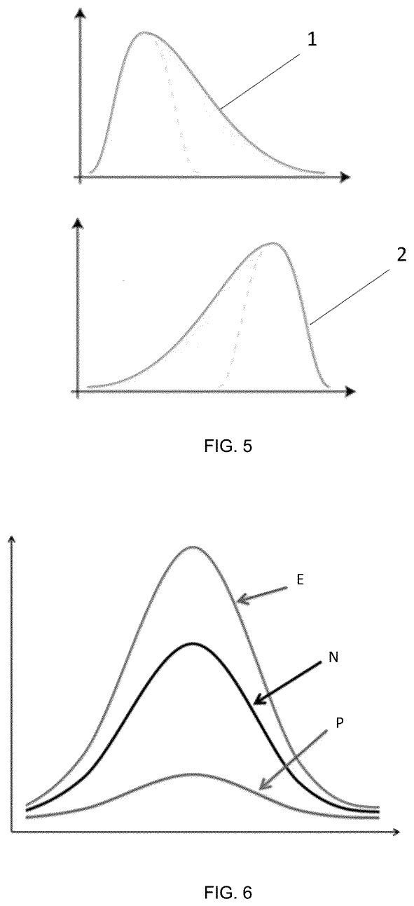

A third marker Mᵢ calculated on the discrete wavelet transform is the measurement of the Fischer asymmetry coefficient. This coefficient may also be called “Skewness”. This coefficient makes it possible to estimate the asymmetry of a given curve. FIG. 5 illustrates two asymmetry measurements on two curves given as examples. Curve 1 is a curve tending to the left and curve 2 is a curve tending to the right. The Fischer asymmetry coefficient has a positive value when the curve tends to the left. This is the case for curve 1. The Fischer asymmetry coefficient has a negative value when the curve tends to the right. This is the case in FIG. 2. Specifically, the Fischer asymmetry coefficient is calculated on the sum of the coefficients over several levels of the discrete wavelet decomposition. Typically, the sum of the coefficients is made between 64 Hertz and 1024 Hertz, i.e. on the four levels of this frequency band.

A fourth marker Mᵢ calculated on the discrete wavelet transform is the measurement of the number of local minima chains of said decomposition. Specifically, a local minima chain is the presence on several discrete wavelet decomposition levels of a same minimum. By measuring the number of minima that are found in each decomposition level, the number of local minima chains is measured. Typically, the measurement is made between 64 Hertz and 1024 Hertz, i.e. on the four levels of this frequency band. The minima repeating in the 64 Hertz to 128 Hertz bands, then 128 Hertz to 256 Hertz, then 256 Hertz to 512 Hertz and finally 512 Hertz to 1024 Hertz are therefore sought. Alternatively, the measurement of the number of local minima chains may be carried out on the continuous wavelet decomposition.

Another signal marker Mᵢ that may be chosen is the measurement on the continuous wavelet decomposition of the number of local maxima chains. In concrete terms, a local maxima chain is the presence on several discrete wavelet decomposition levels of a same maximum. By measuring the number of maxima that are found in each decomposition level, the number of local maxima chains is measured. Typically, the measurement is made between 64 Hertz and 1024 Hertz, i.e. on the four levels of this frequency band. The maxima repeating in the 64 Hertz to 128 Hertz bands, then 128 Hertz to 256 Hertz, then 256 Hertz to 512 Hertz and finally 512 Hertz to 1024 Hertz are therefore sought. Alternatively, the number of local maxima chains may be measured on the discrete wavelet decomposition.

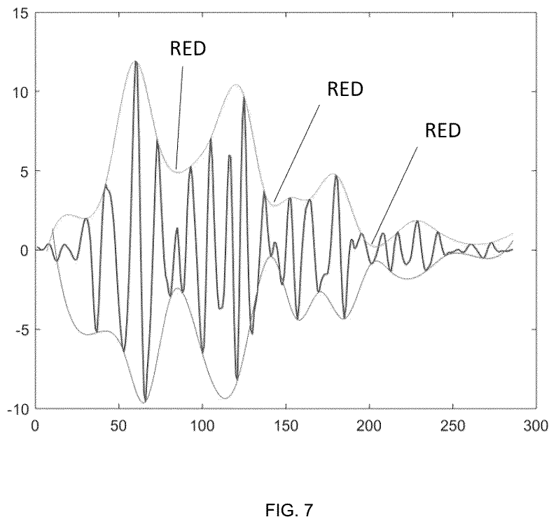

The following two signal makers Mᵢ are measured on the wavelet of the signal comprised in the frequency band ranging from 256 Hertz to 512 Hertz of the signal. The first concerns the measurement of the Kurtosis index Sₖᵤ on this wavelet. “Kurtosis Sₖᵤ” is taken to mean the same indicator as described previously in the application. The second signal marker Mᵢ measured on this wavelet is the measurement of the number of reduced amplitude areas RED of the wavelet. To calculate the number of reduced amplitude areas, the upper and lower signal envelopes are created. Thus, the number of areas of reduced amplitude is calculated on the signal envelopes. This is illustrated by FIG. 7 which shows a signal and the reduced amplitude areas RED detected.

The following three signal markers Mᵢ are measured on the wavelet of the signal comprised in the frequency band ranging from 128 Hertz to 256 Hertz of the signal. The first concerns the measurement of the Kurtosis index Sₖᵤ on this wavelet. Kurtosis Sₖᵤ is taken to mean the same indicator as that described previously in the application. The second signal marker Mᵢ measured on this wavelet is the measurement of the number of areas of reduced amplitude of the wavelet. The number of areas of reduced amplitude is calculated in the same way as for the marker concerning the wavelet of the signal in the frequency range 256 Hertz to 512 Hertz of the signal. The third signal marker concerns the measurement of the RMS of the wavelet in the frequency band 128 Hertz to 256 Hertz. RMS, or Root Mean Square, is taken to mean the measurement of the effective amplitude of the signal.

Finally, a signal marker Mᵢ that may be selected is measured on the wavelet of the signal comprised in the frequency band ranging from 64 to 128 Hertz. This signal marker Mᵢ relates to the measurement of the RMS (Root Mean Square) effective amplitude of the signal.

Other signal markers Mᵢ may be used beyond the fourteen signal markers Mᵢ described. For example, signal markers Mᵢ may be used which are combinations of signal markers Mᵢ already described.

Statistical modality Each first electrophysiological descriptor Dᵢ is associated with a statistical modality MSᵢ. Statistical modality MSᵢ is taken to mean a modality for processing the different quantities measured in order to calculate a value for each descriptor Dᵢ.

It may be recalled that for each descriptor Dᵢ an area of the body of the patient Zᵢ, a signal type Tᵢ (therefore the way in which the individual signals of each electrode EL are captured and used), and a signal marker Mᵢ are selected. It should be specified that once these three choices have been made, the signal marker Mᵢ for the selected signal type is measured for each electrode of the body area of the patient Zᵢ available in said selected area Zᵢ. Thus, for each descriptor Dᵢ, a plurality of measurements of the value of the signal marker Mᵢ is obtained. Using the statistical modality MSᵢ makes it possible to transform this plurality of values into a final value for the selected descriptor Dᵢ For each descriptor Dᵢ, a statistical modality MSᵢ may be selected from a set of available statistical modalities MSᵢ.

A first statistical modality is the fifth percentile minimum of the measured values. It will be recalled that, for the values, all the values measured on each electrode in the predefined area of the body of the patient Zᵢ are taken. For this statistical modality, the five percent of the lowest values are removed from all the measured values and the minimum value is selected from the remaining values. This statistical modality has the advantage, by removing the five percent of the lowest values, of eliminating outliers that could distort the representativeness of the measurement.

A second statistical modality that can be selected is the 95th percentile maximum. For this statistical modality, the five percent of the highest values are taken from all the selected values. The highest value of the remaining values is then selected. This statistical modality makes it possible not to take into account, for a measurement of a maximum value, the outliers which could appear in the highest values measured. In this way, there is an upper limit representative of all the measured values.

A third statistical modality MSᵢ is the average of the measured values. The average is a conventional indicator and representative of a distribution.

A fourth statistical modality MSᵢ is the standard deviation calculated on the set of measured values. The standard deviation is a representative value of the dispersion of the values. In our case, the dispersion may be a significant value, a large variance in the measurements being able to be the sign of a disorder in the patient's cardiac activity.

A fifth statistical modality MSᵢ that may be selected is the median. The median value of a set of values is the value making it possible to separate all of the values into two sets of the same size. This value provides a teaching which may vary from that given by the average value, because the median makes it possible not to give too much importance to outliers close to the maximum and minimum of the values measured.

A sixth statistical modality MSᵢ is the value of the interquartile. To calculate this value, the value of the 25th percentile and the value of the 75th percentile are calculated. The value of the interquartile represents the difference between the value of the 75th percentile and the value of the 25th percentile. The interquartile is an interesting statistical value to look at to characterize the distribution of the measured values.

Calculation of a Score

Once the subset {Dₖ} of the electrophysiological descriptors is selected, a calculation of the value of each descriptor of the subset {Dₖ} is performed.

In order to be able to use the value of each descriptor Dᵢ, a threshold value V_{threshold} is available for each of them. Each threshold value V_{threshold} for each descriptor Dᵢ of the subset {Dₖ} may advantageously be defined by the input parameter Inp.

Each value of each descriptor Dᵢ of the subset {D} is compared with the threshold value V_{threshold} of said descriptor Dᵢ. Threshold value V_{threshold} is taken to mean a lower or upper threshold value. Thus, depending on the threshold value V_{threshold}, the threshold may be exceeded either if the value of the descriptor is lower than the threshold value, or if the value of the descriptor is greater than the threshold value V_{threshold}.

Alternatively or additionally, a descriptor Dᵢ comprises several different threshold values V_{threshold}. For example, a descriptor Dᵢ may comprise an upper threshold value V_{threshold} and a lower threshold value V_{threshold}. Thus, the threshold is exceeded if the value of the descriptor is between the lower threshold value V_{threshold} and the upper threshold value V_{threshold}. Similarly, for a descriptor, the threshold may be exceeded when the value of said descriptor is lower than the lower value V_{threshold} or when the value is greater than the upper value V_{threshold}. Similarly, a descriptor Dᵢ may comprise three or more threshold values V_{threshold} which define value ranges corresponding to the exceeding for the value of the descriptor Dᵢ.

According to one embodiment, the threshold value V_{threshold} is defined by the scientific literature relating to representative cardiac data.

For example, for a descriptor Dᵢ that relates to the upper right zone Zᵢ of the patient's torso, according to a type of unipolar signal Tᵢ, on which the signal marker Mᵢ is the duration of depolarization of the ventricles (QRS), with a statistical modality STi that is the average of the values measured on each electrode of the predefined area Zᵢ, the threshold value may be set at 120 milliseconds. Thus, in this case, it is considered that the threshold value of the descriptor is exceeded for a duration greater than 120 milliseconds. In this example, the score retained is 0 if the value is less than 120 ms and the score is 2 if the value is greater than 120 ms. According to one example, different thresholds may be defined for a descriptor Dᵢ associated with the marker and the statistical modality. These different thresholds allow different scores to be assigned, for example an intermediate threshold of 80 ms allows a first score of 0 to be defined if the value is less than 80 ms, a score of 1 if the value is between 80 ms and 120 ms and a score of 2 if the value is greater than 120 ms.

According to another example, for a descriptor Dᵢ which concerns the entire torso of the patient Z₅, according to a unipolar signal type Tᵢ, on which the signal marker Mᵢ is the absolute amplitude between 40 and 250 Hz of the last 40 milliseconds of the QRS (measurement which corresponds to the measurement of late potentials of the QRS), with a statistical modality which is the average of the values measured on each electrode of the predefined area Zᵢ, the threshold value can be set at 20 microvolts. Thus, in this case, it is considered that the threshold value of the descriptor is exceeded for a measured tension value which is less than 20 microvolts. In this example, a score of 0 may be assigned to the value above 20 microvolts and a score of 1 may be assigned to the value below 20 microvolts.

More broadly, many examples of threshold values are described in the publication entitled “Caractérisation et détection des micro-potentiels anormaux associés aux arythmies ventriculaires létales, par analyse des signaux électrocardiographiques haute-ŕesolution enregistrés à la surface du torse” (Characterization and detection of abnormal micro-potentials associated with lethal ventricular arrhythmias, by analysis of high-resolution electrocardiographic signals recorded on the surface of the torso) (Nolwenn TAN et al.).

According to different embodiments, the thresholds may correspond to physiological signs characterizing an electrical activity, a physical effort or a medical history. According to one example, the threshold values may be chosen so as to identify an index of the presence of a given cardiac condition or singular cardiac electrical activity.

The comparison between the value of each descriptor Dᵢ of the subset {Dₖ} and the respective threshold value V_{threshold} of each descriptor Dᵢ is performed. The result of these comparisons makes it possible to establish a score. The established score defines an electrophysiological parameter. More precisely, the score takes into account the number of times the value of one of the descriptors Dᵢ of the subset {Dₖ} exceeds the threshold value V_{threshold}. The number of times the threshold value V_{threshold} is exceeded makes it possible to establish the score. The score calculated according to the invention has the interest of taking into account the value of a set of different physiological data, which makes it very representative compared to conventional measurements taking into account only a limited number of parameters.

According to one aspect, the score is equal to the number of values of descriptors Dᵢ exceeding the threshold value V_{threshold} associated with said descriptor Dᵢ. A score established in this manner is very representative of a cardiac activity. If the subset {Dₖ} is chosen correctly, the score is very representative of the patient's cardiac activity.

According to one aspect, the score is compared to a threshold score value. Said threshold score value is specific to the predefined condition ET₁.

Device for Generating the Electrophysiological Parameter

The invention also relates to a device for generating the electrophysiological parameter. The characteristics described above concerning the method according to the invention also apply to the device according to the invention. The characteristics described below for the device also apply to the method according to the invention.

The device for generating an electrophysiological parameter comprises a plurality of electrodes which are arranged on the surface of the patient's body. Each surface electrode defines a channel Vᵢ.

According to one embodiment, the device comprises adhesive strips comprising the surface electrodes. According to this aspect, the adhesive strips are intended to be applied to the surface of the patient's body.

Advantageously, each adhesive strip comprises several surface electrodes. This provision makes it easier to install the electrodes on the patient, the installation of a strip comprising several electrodes being simpler than installing the electrodes one by one.

According to one aspect, the device comprises a vest or jacket comprising the plurality of measuring electrodes EL. The vest is intended to be put on by the patient. This provision enables a rapid installation of the device on the patient. According to one example, the device comprises at least 14 electrodes.

The device comprises a means of measuring the signal of each channel Vᵢ. More precisely, the measurement means is configured to measure an electrical potential of each of the channels Vᵢ. For example, the measurement means may be an acquisition card. The acquisition card may comprise an input to collect an electrical signal, and an analog digital converter to digitize the acquired signal. The digitized signal is then transmitted to a calculator. For example, the digitized signal may be transmitted to a computer that performs the processing steps on the signal.

The device comprises a means of calculation. The calculation means records the measurements of channels Vᵢ supplied by the measurement means. The calculator then processes this data.

The calculator selects a subset {Dₖ} of descriptors Dᵢ from the set {D_{N}} of descriptors Dᵢ. This selection is made according to the input parameter Inp.

The calculator then calculates the value of each descriptor Dᵢ of the subset {Dₖ}. This calculation is performed from measurements of channels Vᵢ. The calculation is performed according to the predefined area Zᵢ, signal type Tᵢ, signal marker MSᵢ and statistical modality STᵢ selected for the descriptor Dᵢ in question.

The calculator then compares the value of the selected descriptors Dᵢ with the threshold value V_{threshold} relative to each descriptor Dᵢ.

The calculator then calculates a score based on comparisons of the estimated values of the descriptors Dᵢ with their threshold value V_{threshold}. The score is calculated as described above.

According to one embodiment, the device comprises a device for detecting the respiration phases of the patient. Such a device detects when the patient is in the expiration phase or “flat” respiration phase. It also detects when the patient is in the inspiration phase. Respiration tends to interfere with the measurements carried out at the level of the electrodes EL. This is notably the case during inspiration phases during which heart beats and their measurement may be affected. Preferably, the measurement of the potential of each channel Vᵢ is performed during the expiration phase. This provision makes it possible to avoid disturbances caused by a measurement during inhalation phases. The device for detecting respiration phases may be connected to the calculator. Alternatively, it is connected to the means of measuring the signal of each channel Vᵢ.

According to one embodiment, the device for detecting respiration phases is a plethysmography belt. The plethysmography belt is a practical way to perform this type of detection.

An object of the invention is also a method for generating an electrophysiological parameter which comprises:
selection of a subset of electrophysiological descriptors from a set of predefined electrophysiological descriptors according to an input parameter defining a measurement context, each electrophysiological descriptor of the subset being associated with at least one channel, a signal type, a signal marker and a statistical modality for calculation;
arrangement of a plurality of surface electrodes on a patient's body;
recording of a plurality of cardiac electrical activities defining said channels, each channel being obtained by the recordings of at least two electrodes;
estimation of the set of electrophysiological descriptors of the subset, each electrophysiological descriptor being calculated from the statistical modality that is applied to the signal marker of the acquired signal according to the signal type on a selected channel associated with said electrophysiological descriptor;
comparison of the value of the set of electrophysiological descriptors with at least one threshold value specific to the set of electrophysiological descriptors, said at least one threshold value being defined by a statistical distribution of said descriptors of a set of patients of a reference group;
display of a result of the comparison step and a classification of all or part of the results of the comparison step according to a sorting key.

According to one embodiment, the sorting key takes into account an increasing or decreasing order of incremental values. Alternatively or additionally, the sorting key takes into account the value of the differences recorded between the value of each descriptor and the at least one threshold value relative to said descriptor. Alternatively or additionally, the sorting key takes into account the descriptors, i.e. it classifies the descriptors and their value according to a predefined order. Alternatively or additionally, the sorting key takes into account the area on the torso of the patient considered in the choice of the descriptors. Alternatively or additionally, the sorting key takes into account the descriptors.

Optionally, the display step comprises a step of displaying a calculated score defining an electrophysiological parameter as a function of the exceeding of at least one threshold value defined by the statistical distribution.

### Nomenclature

Dᵢ: Electrophysiological descriptor
{Dₖ}: Subset of electrophysiological descriptors
{D_{N}}: Set of electrophysiological descriptors
Inp: Input parameter
Vᵢ: Channel
Zᵢ: Predefined area of the patient's body
Z₁: Upper right area of the patient's torso
Z₂: Upper left area of the patient's torso
Z₃: Lower right area of the patient's torso
Z₄: Lower left area of the patient's torso
Z₅: Area covering the totality of the patient's torso
Tᵢ: Signal type
Mᵢ: Signal marker
MSᵢ: Statistical modality for calculation
EL: Electrode
EL₁: Central electrode
ELₗₐₚ: Laplacian electrode
By: Vertical bipole
Bh: Horizontal bipole
DISPO: arrangement of a plurality of electrodes
ENR: Recording of a plurality of electrical activities
EST: Estimation of descriptors
COMP: Comparison of the value of a descriptor with a threshold value
V_{threshold}: Threshold value
CALC: Calculation of a score
Sₖᵤ: Kurtosis
N: Curve representing a normal distribution
P: Flat curve
E: Slender curve
1: Curve tending to the left
2: Curve tending to the right
RED: Reduced amplitude range

1. A method for generating an electrophysiological parameter, the method comprising:
selecting a subset of first electrophysiological descriptors from a set of first predefined electrophysiological descriptors, according to an input parameters defining a measurement context, each first electrophysiological descriptor of the subset being associated with at least one channel, a signal type, a signal marker and a statistical modality for calculation; at least one first descriptor of the subset being associated with a statistical modality different to that of another first descriptor of the subset and a signal marker different to that of the other first descriptor;
arranging a plurality of surface electrodes on a patient's body;
recording of a plurality of cardiac electrical activities defining said channels, each channel being obtained by the recordings of at least two electrodes;
estimating the set of electrophysiological descriptors of the subset, each electrophysiological descriptor being calculated from the statistical modality that is applied to the signal marker of the acquired signal according to the signal type on a selected channel associated with said electrophysiological descriptor;
comparing the value of the set of electrophysiological descriptors with at least one threshold value specific to the set of electrophysiological descriptors, said at least one threshold value being defined by a statistical distribution of said descriptors of a set of patients of a reference group, and
calculating a score defining an electrophysiological parameter as a function of the exceeding of the at least one threshold value.

2. The method for generating an electrophysiological parameter according to claim 1, wherein the comparing is performed by comparing the value of the set of descriptors with the threshold value defined by a statistical distribution of said descriptors of the set of patients of the reference group of dimension equal to the number of descriptors of the subset.

3. The method for generating an electrophysiological parameter according to claim 1, wherein:
the comparing is performed by comparing the value of each electrophysiological descriptor with at least one threshold value specific to said electrophysiological descriptor, said at least one threshold value being defined by a statistical distribution of said descriptor of a set of patients of the reference group, and
calculating the score defining the electrophysiological parameter is performed by incrementing said score each time an electrophysiological descriptor exceeds the at least one threshold value specific to it.

4. The method for generating an electrophysiological parameter according to claim 1, wherein the subset comprises at least one geographical descriptor which is associated with several channels and several geographical groups, each geographical group being formed by a central channel and the at least four channels adjacent to the central channel, the value of the electrophysiological descriptor being determined:
by comparing the value, for each geographical group, of the measurement of each channel according to the signal type and the signal marker selected with at least one geographical threshold value specific to said electrophysiological descriptor and said channel; and
by counting the number of geographical groups for which the value of at least three channels exceeds the geographical threshold value that is specific to it.

5. The method according to claim 1, wherein the input parameter defining the measurement context defines a subset of characteristic descriptors:
of a given cardiac pathology and/or;
of a patient profile comprising at least one age and/or gender/sex.

6. The method according to claim 1, wherein each first electrophysiological descriptor of the subset is associated with at least one channel of a predefined area of the patient's body from a set of predefined areas and wherein for each electrophysiological descriptor, the predefined area on the patient's body is chosen from:
an upper right area of the torso;
an upper left area of the torso;
a lower right area of the torso;
a lower left area of the torso; and
an entire torso of the patient.

7. The method according to claim 1, wherein for each electrophysiological descriptor, the signal type analyzed is chosen from:
a unipolar signal taken between an electrode of the chosen body area and a reference electrode;
a vertical bipolar signal taken between two electrodes of the given area, one of the two electrodes being offset along a vertical line leading from the feet to the head of the patient with respect to the other electrode;
a horizontal bipolar signal taken between two electrodes of the given area, one of the two electrodes being offset along a horizontal line going from one arm to the other of the patient with respect to the other electrode; and
a Laplacian signal estimated by subtracting from the potential of a central electrode the average tension of the eight electrodes directly adjacent to said central electrode.

8. The method according to claim 1, wherein for at least one electrophysiological descriptor, the signal marker is the measurement of the voltage of an averaged signal.

9. The method according to claim 1, wherein for at least one electrophysiological descriptor, the marker is the measurement, on the averaged and filtered signal between 40 and 250 Hertz, of the duration of depolarization of the ventricles or fragmentation of the signal during depolarization of the ventricles.

10. The method according to claim 1, wherein for at least one electrophysiological descriptor, the signal marker is the measurement on the discrete wavelet decomposition of the signal:
of the energy of the sum of the wavelets;
of the Kurtosis;
of the Fisher asymmetry coefficient; or
of the number of local minima.

11. The method according to claim 1, wherein for at least one electrophysiological descriptor, the signal marker is the measurement on the continuous wavelet decomposition of the signal of the number of chains of local maxima.

12. The method according to claim 1, wherein for at least one descriptor, the signal marker is:
either the measurement on the wavelet taken between 256 and 512 Hertz of the signal:
i. of the Kurtosis; or
ii. of the number of areas with reduced amplitudes;
or the measurement on the wavelet taken between 128 and 256 Hertz of the signal:
iii. of the Kurtosis;
iv. of the number of areas of reduced amplitude; or
v. of the RMS (Root Mean Square);
or the measurement on the wavelet taken between 64 and 128 Hertz of the RMS (Root Mean Square).

13. The method according to claim 1, wherein for at least one electrophysiological descriptor, the statistical modality is chosen from:
the fifth percentile minimum of the measured values of the signal on each electrode of the predefined area;
the ninety fifth percentile maximum of the measured values of the signal on each electrode of the predefined area;
the average of the measured values of the signal on each electrode of the predefined area;
the standard deviation of the measured values of the signal on each electrode of the predefined area;
the median of the measured values of the signal on each electrode of the predefined area; and
the interquartile of the measured values of the signal on each electrode of the predefined area.

14. A device for generating an electrophysiological parameter, the device comprising:
a plurality of surface electrodes configured to be deposited on a patient's body and to measure an electrical potential of the surface of the patient's body, each surface electrode defining a channel;
a means of measuring the signal of each channel;
a calculation means configured to:
select a subset of descriptors from a set of predefined descriptors according to an input parameter defining a measurement context, each electrophysiological descriptor of the subset being associated with at least one channel, a signal type, a signal marker and a statistical modality for calculation; at least one descriptor of the subset being associated with a statistical modality different to that of a second descriptor of the subset and a signal marker different to that of the second descriptor;
record a plurality of cardiac electrical activities defining said channels, each channel being obtained by the recordings of at least two electrodes;
estimate the set of electrophysiological descriptors of the subset, each electrophysiological descriptor being calculated from the statistical modality that is applied to the signal marker of the acquired signal according to the signal type on a selected channel associated with said electrophysiological descriptor;
compare the value of the set of electrophysiological descriptors with at least one threshold value specific to the set of electrophysiological descriptors, said at least one threshold value being defined by a statistical distribution of said descriptors of a set of patients;
calculate a score defining an electrophysiological parameter as a function of the exceeding of the at least one threshold value defined by the statistical distribution.

15. The device for generating an electrophysiological parameter according to claim 14, further comprising a device for detecting a patient's respiration phases.

16. A device for generating an electrophysiological parameter, comprising a plurality of electrodes, a receiver of signals measured by the electrodes, a memory for recording the measured data and a calculator making it possible to perform operations and processings on the measured data, said device comprising means configured to implement the method of claim 1.

17. The device according to claim 15, wherein the device for detecting a patient's respiration phases is a plethysmography belt.
